SEQUENCE LISTING (9 sequences)

SEQ ID NO: 1 - DNA, 1398 bp, Candida parapsilosis
  CDS (1)..(1398)
atgcgttactttgctattgctttcttgctcatcaataccatttcagcttttgtcttggctcccaaaaagccatctcaagacgatttctacactccaccacaaggttatgaagctcaacctcttggttctattttgaaaacaagaaacgtccccaatccattgactaatgttttcactccagttaaagttcaaaatgcatggcaattattggttagatctgaagatacatttggtaacccaaacgccatagtcactaccattattcaacctttcaatgctaaaaaggataagcttgtttcttatcaaacatttgaagattctggtaaattggattgtgctccatcatatgctattcaatatggatcggacatttcgactttgaccactcaaggtgaaatgtactacatctctgctttattagatcaaggttactatgttgtcactcctgattacgagggtccaaagagtacattcactgtagggttgcaatcaggaagagctactttgaattcgcttagagctactttgaaatcaggaaacttgactggtgtttcatcagacgctgagacattattgtggggttattcaggaggaagtcttgcttcaggatgggctgctgctatacaaaaagaatatgctccagagttgagtaaaaacttgcttggtgctgcacttggtggattcgttacaaacattactgccactgctgaagctgttgatagtggtccatttgcaggaatcatctccaatgcattggctggtattggaaatgaataccctgatttcaaaaactatcttttgaaaaaagtgtcaccattgctttcaatcacttatcgtttgggaaacactcactgtttgcttgatggtggtattgcttatttcggtaaatcattcttttccagaattattagatatttccctgatggatgggatcttgtcaaccaagaacctatcaaaaccatcttgcaagataatggattggtttaccaaccaaaggacttgaccccacaaattccattattcatctaccacggtaccttggatgcaattgtccccattgtcaactcaagaaagacattccaacaatggtgtgattggggactcaaatctggtgaatataatgaagatttgaccaatggacacattactgaatcaattgtgggtgcaccagctgctttgacttggattatcaatcgtttcaatggacagcctccagttgatggatgtcaacataatgtgagagcttcaaacttggaatatccaggaactccacaatcaatcaagaattactttgaagctgcattgcacgcaattttgggctttgatttgggtccagatgttaagagagataaggttactttgggcggattgctcaagttggaacgttttgctttttag

SEQ ID NO: 2 - protein, 465 aa, Candida parapsilosis
MRYFAIAFLLINTISAFVLAPKKPSQDDFYTPPQGYEAQPLGSILKTRNVPNPLTNVFTPVKVQNAWQLLVRSEDTFGNPNAIVTTIIQPFNAKKDKLVSYQTFEDSGKLDCAPSYAIQYGSDISTLTTQGEMYYISALLDQGYYVVTPDYEGPKSTFTVGLQSGRATLNSLRATLKSGNLTGVSSDAETLLWGYSGGSLASGWAAAIQKEYAPELSKNLLGAALGGFVTNITATAEAVDSGPFAGIISNALAGIGNEYPDFKNYLLKKVSPLLSITYRLGNTHCLLDGGIAYFGKSFFSRIIRYFPDGWDLVNQEPIKTILQDNGLVYQPKDLTPQIPLFIYHGTLDAIVPIVNSRKTFQQWCDWGLKSGEYNEDLTNGHITESIVGAPAALTWIINRFNGQPPVDGCQHNVRASNLEYPGTPQSIKNYFEAALHAILGFDLGPDVKRDKVTLGGLLKLERFAF

SEQ ID NO: 3 - protein, 449 aa, Candida parapsilosis
FVLAPKKPSQDDFYTPPQGYEAQPLGSILKTRNVPNPLTNVFTPVKVQNAWQLLVRSEDTFGNPNAIVTTIIQPFNAKKDKLVSYQTFEDSGKLDCAPSYAIQYGSDISTLTTQGEMYYISALLDQGYYVVTPDYEGPKSTFTVGLQSGRATLNSLRATLKSGNLTGVSSDAETLLWGYSGGSLASGWAAAIQKEYAPELSKNLLGAALGGFVTNITATAEAVDSGPFAGIISNALAGIGNEYPDFKNYLLKKVSPLLSITYRLGNTHCLLDGGIAYFGKSFFSRIIRYFPDGWDLVNQEPIKTILQDNGLVYQPKDLTPQIPLFIYHGTLDAIVPIVNSRKTFQQWCDWGLKSGEYNEDLTNGHITESIVGAPAALTWIINRFNGQPPVDGCQHNVRASNLEYPGTPQSIKNYFEAALHAILGFDLGPDVKRDKVTLGGLLKLERFAF

SEQ ID NO: 4 - protein, 22 aa, Artificial Sequence
  MISC_FEATURE (6)..(7): Xaa is any essential or unessential amino acid
  MISC_FEATURE (9)..(10): Xaa is any essential or unessential amino acid
  MISC_FEATURE (13)..(18): Xaa is any essential or unessential amino acid
GYSGGXXAXXWAXXXXXXYAPE

SEQ ID NO: 5 - protein, 22 aa, Artificial Sequence
  MISC_FEATURE (6)..(7): Xaa is any essential or nonessential amino acid
  MISC_FEATURE (9)..(10): Xaa is any essential or nonessential amino acid
  MISC_FEATURE (13)..(18): Xaa is any essential or nonessential amino acid
GYSGGXXAXXWAXXXXXXYAPD

SEQ ID NO: 6 - protein, 22 aa, Artificial Sequence
  MISC_FEATURE (6)..(7): Xaa is any essential or nonessential amino acid
  MISC_FEATURE (9)..(10): Xaa is any essential or nonessential amino acid
  MISC_FEATURE (13)..(18): Xaa is any essential or nonessential amino acid
GFSGGXXAXXWAXXXXXXYAPE

SEQ ID NO: 7 - protein, 22 aa, Artificial Sequence
  MISC_FEATURE (6)..(7): Xaa is any essential or nonessential amino acid
  MISC_FEATURE (9)..(10): Xaa is any essential or nonessential amino acid
  MISC_FEATURE (13)..(18): Xaa is any essential or nonessential amino acid
GFSGGXXAXXWAXXXXXXYAPD

SEQ ID NO: 8 - protein, 20 aa, Artificial Sequence
  MISC_FEATURE (6)..(7): Xaa is any essential or nonessential amino acid
  MISC_FEATURE (9)..(10): Xaa is any essential or nonessential amino acid
  MISC_FEATURE (13)..(18): Xaa is any essential or nonessential amino acid
GYSGGXXAXXWAXXXXXXYA

SEQ ID NO: 9 - protein, 20 aa, Artificial Sequence
  MISC_FEATURE (6)..(7): Xaa is any essential or nonessential amino acid
  MISC_FEATURE (9)..(10): Xaa is any essential or nonessential amino acid
  MISC_FEATURE (13)..(18): Xaa is any essential or nonessential amino acid
GFSGGXXAXXWAXXXXXXYA

(19) United States
(12) Patent Application Publication
(10) Pub. No.: US 2007/0244021 A1
(43) Pub. Date: Oct. 18, 2007

(54) Polypeptides with perhydrolase activity

(76) Inventors: Eric Dubreucq, Montpelier (FR); Albrecht Weiss, Langenfeld (DE); Guy Moulin, Montferrier-sur-Lez (FR)

Correspondence Address: COGNIS CORPORATION;PATENT DEPARTMENT, 300 BROOKSIDE AVENUE, AMBLER, PA, 19002, US

(21) Appl. No.: 11/709,604

(22) Filed: Feb. 22, 2007

(30) Foreign Application Priority Data
Feb. 23, 2006 (EP) 06003668

(51) Int. Cl. C11D 1/32; C07H 21/04; C12N 1/00; C12N 15/63; C07K 14/40

(52) U.S. Cl. 510/367; 435/243; 435/320.1; 530/326; 530/350; 536/23.74

(57) ABSTRACT

The invention relates to polypeptides having perhydrolase activity with an amino acid sequence which is at least 80% homologous or at least 65% identical to the amino acid sequence shown in SEQ ID No. 3, with the exception of SEQ ID NO. 3. The invention also relates to polypeptides having perhydrolase activity which contain at least one motif which is at least 50% homologous or at least 70% identical to a motif selected from the group consisting of SEQ ID NO. 4: GYSGGxxAxxWAxxxxxxYAPE, SEQ ID NO 5: GYSGGxxAxxWAxxxxxxYAPD, SEQ ID NO 6: GFSGGxxAxxWAxxxxxxYAPE, SEQ ID NO 7: GFSGGxxAxxWAxxxxxxYAPD, SEQ ID NO 8: GYSGGxxAxxWAxxxxxxYA and SEQ ID NO 9: GFSGGxxAxxWAxxxxxxYA.

### FIELD OF THE INVENTION

This invention relates to polypeptides having perhydrolase activity, to processes for their production, to care, cleaning, bleaching or disinfecting preparations containing these polypeptides with perhydrolase activity and to their use. The present invention also relates to the nucleic acids which code for these polypeptides.

### PRIOR ART

Enzymes are being increasingly used as catalysts in chemical and biochemical synthesis. In many cases, esterases, especially lipases (EC 3.1.1.3), are already being used in industrial processes for lipolysis, esterification and transesterification by virtue of the often milder reaction conditions. Enzymes have long been used in laundry detergents or cleaning products in order to obtain or improve cleaning effects. The enzymes used in this connection include above all proteases, amylases and esterases, especially lipases.

Inorganic, highly alkaline hydrogen peroxide sources, such as percarbonate or perborate, are used in combination with bleach boosters (TAED or NOBS) for the conventional chemical bleaching of laundry. However, this standard bleaching system is only fully effective at temperatures of 50° C. to 60° C. No optimal bleaching system is available for the low temperature range. In addition, local spotting can occur in colored fabrics. Above all, however, a local overconcentration of bleach booster dissolving in the immediate vicinity of fabrics can result in oxidation damage. The bleach component, hydrogen peroxide, is formed through spontaneous decomposition of the salt and thus leads quickly, but briefly, to high concentrations. Gentle, delayed bleaching at a milder pH is not possible. There is also no optimal bleaching system available for use in weakly basic matrixes, for example liquid formulations.

Relatively long-chain peracids are suitable for effective bleaching, even at temperatures of 30° C. to 40° C. In view of their lack of stability, peracids for bleaching cannot be directly incorporated in the detergent formulation and have to be produced in situ. The bleach boosters mentioned above are used for this purpose. In combination with hydrogen peroxide for example, TAED releases peracetic acid.

The enzymatic production of peracids has been known for some time. The use of these peracids has been described for bleaching and disinfection in laundry detergents, cleaning preparations or the like. The enzymes used are capable of carrying out perhydrolysis reactions starting from esters. The technical enzymes suitable for this purpose include lipases, perhydrolases, acyl transferases, esterases and other hydrolases. Besides the desired perhydrolysis reaction, these enzymes also show their natural hydrolysis reactions. The effect of this is that the ester used is consumed in unwanted reactions and a very low ratio of perhydrolysis to hydrolysis is observed. This means that the desired formation of peracids is suppressed in favor of the formation of acids. This has the effect that a higher concentration of ester has to be used to achieve an adequate concentration of peracid which, in turn, leads to high costs, formulation problems and other disadvantages.

The described enzyme systems lead to the formation of peracids. Normally, these enzymes also have the ability to hydrolyze peracids to the corresponding acids, i.e. to catalyze a back-reaction of the product formed. Thus, the enzymes in question may be selectively used for reducing or destroying peracids. In the described applications, however, there is a need for a relatively high concentration of peracid and hence for slower hydrolysis of the peracids. That this is the case is shown by the existence of the peracid in the system used.

These enzymes, which are used for forming the peracids, are directly added to the laundry detergent or to the cleaner.

WO 2005/056782 describes a perhydrolase from M. smegmatis which is used in laundry detergents or other cleaning or bleaching systems.

There is still a need for enzyme systems for the described applications, for improved methods for developing such systems and for improved cleaning, disinfecting, oxidizing or bleaching preparations. These preparations should be inexpensive, effective, above all at low temperatures, mild and easy to handle.

Accordingly, the problem addressed by the present invention was to find an enzyme system which would have a high affinity for hydrogen peroxide by comparison with water, so that hydrolysis would be a secondary reaction by comparison with perhydrolysis and the enzyme/hydrogen peroxide/ester systems could be used more effectively and there would only be limited catalysis of the peracid back reaction.

### BRIEF DESCRIPTION OF THE DRAWINGS

FIG. 1 is a phylogentic tree of Candida parapsilosis CBS 604 perhydrolase (CpLIP2) and other related sequences.

FIG. 2 is a diagram depicting the fold structure of the perhydrolase from Candida parapsilosis.

### DESCRIPTION OF THE INVENTION

The present invention relates to polypeptides having perhydrolase activity with an amino acid sequence which has a homology of at least 80% to the amino acid sequence shown in SEQ ID NO. 3 with the exception of SEQ ID NO. 3. Polypeptides having perhydrolase activity of which the amino acid sequence has a homology to the amino acid sequence shown in SEQ ID NO. 3 of at least 83%, preferably at least 85%, more preferably at least 90%, most preferably at least 95%, 96%, 96.5%, 97%, 97.5%, 98%, 98.5%, 99% and, in one most particularly preferred embodiment, 99.5% are particularly preferred.

The present invention also relates to polypeptides having perhydrolase activity with an amino acid sequence which has an identicality of at least 65% to the amino acid sequence shown in SEQ ID NO. 3 with the exception of SEQ ID NO. 3. Polypeptides having perhydrolase activity of which the amino acid sequence has an identicality to the amino acid sequence shown in SEQ ID NO. 3 of at least 70%, at least 75%, at least 80%, preferably at least 85%, more preferably at least 95%, 96%, 96.5%, 97%, 97.5%, 98%, 98.5%, 99% and, in one most particularly preferred embodiment, 99.5% are particularly preferred.

In the context of the invention, homology in relation to the amino acid sequence is understood to be variations in individual amino acids in the sequence which do not alter the function of the polypeptide. Accordingly, a polypeptide having at least 80% homology has a variable amino acid exchange in at most 19.9% of the positions equivalent to SEQ ID NO. 3 and, in 80% of the positions equivalent to SEQ ID NO. 3 in the amino acid sequence, either identical amino acids or amino acids with the same function, charge or hydrophobicity which are selected so that the resulting polypeptide has the same or improved perhydrolase activity. The homology determination is carried out by a structure comparison with alignments using computer programs and special algorithms, such as preferably BLAST, CLUSTAL X, ALIGN, PASTA, FASTA, PILEUP, BESTFIT, GAP. According to the invention, the alignment is preferably carried out using the BLAST algorithm with the BLOSUM 62 matrix.

Identicality in relation to the amino acid sequence is understood to be an exact sameness of the amino acids in the positions equivalent to the reference amino acid sequence. For a polypeptide having perhydrolase activity with an amino acid sequence which has at least 65% identicality to the amino acid sequence shown in SEQ ID NO. 3, this means that at least 65% of the positions in an amino acid sequence equivalent to the positions in SEQ ID NO. 3 have exactly the same amino acid.

A position in one amino acid sequence is understood to be equivalent to a position in another amino acid sequence when these positions are in accordance, i.e. correspond, through alignment after the sequence comparison.

The methods of alignment are known to the expert and are carried out with computer programs and special algorithms, such as preferably BLAST, CLUSTAL X, ALIGN, PASTA, FASTA, PILEUP, BESTFIT, GAP. According to the invention, the alignment is preferably carried out using the BLAST algorithm with the BLOSUM 62 matrix.

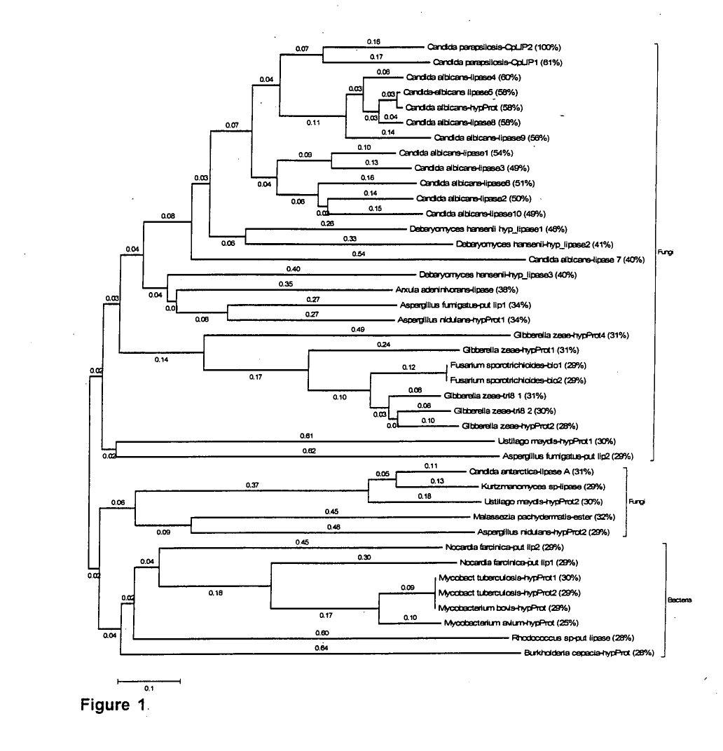

With these programs, it is also possible in some cases to establish phylogenetic trees which are able to represent the degree of relationship of certain enzymes to other enzymes or polypeptides and their identicalties. The phylogenetic tree for SEQ ID NO. 3 is shown in FIG. 1.

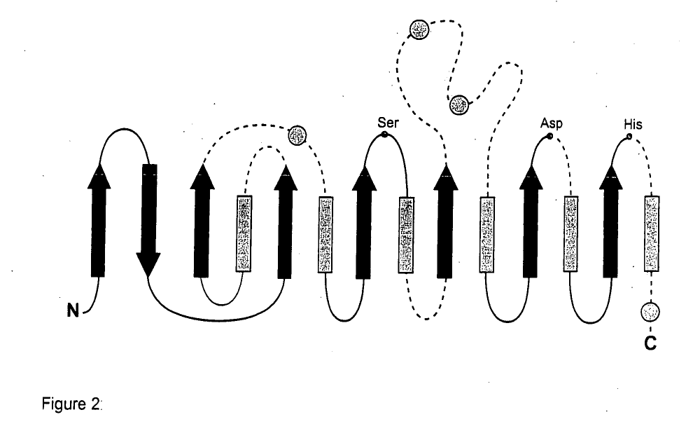

FIG. 2 shows the diagram of the fold structure of the perhydrolase from Candida parapsilosis from the N-terminal to the C-terminal end. In FIG. 2, the gray areas represent the alpha helix of the enzyme, the black arrows correspond to the beta-fold leaf and the characterized amino acids named correspond to the catalytic center. The chain-line links correspond to variable regions and the solid-line links describe the conserved regions.

By comparison with known enzymes deposited, for example, in generally accessible data banks, the enzymatic activity of an enzyme under consideration can be gauged from the amino acid or nucleotide sequence. This can be qualitatively or quantitatively modified by other regions of the protein which are not involved in the actual reaction. This could relate, for example, to enzyme stability, activity, the reaction conditions or to the substrate specificity. Such a comparison is made by assigning similar sequences in the nucleotide or amino acid sequences of the proteins under consideration to one another. This is known as homologization. A tabular association of the particular positions is known as alignment. In the analysis of nucleotide sequences, both complementary strands and all three possible reading frames have to be taken into account, as do the degeneracy of the genetic code and the organism-specific codon usage.

A compilation of all the positions according in the compared sequences is known as a consensus sequence. Such a comparison also enables the similarity of the compared sequences to one another to be stated. This is expressed as percent identicality, i.e. the percentage of identical nucleotides or amino acid residues in the same positions. A broader definition of homology includes the conserved amino acid exchanges in this value and is known as percent homology. Such statements can be made about entire proteins or genes or only about individual regions.

A protein or polypeptide in the context of the present invention is understood to be a substantially linear polymer composed of the natural amino acids which assumes an at least three-dimensional structure to perform its function. In the present application, the 19 proteinogenic, naturally occurring L-amino acids are designated by the internationally accepted 1 and 3 letter codes. The terms polypeptide and polypeptides are used synonymously in the present specification and encompass both the singular and the plural of polypeptides.

An enzyme in the context of the present invention is understood to be a protein which performs a certain biocatalytic function. The amino acid sequence of the enzyme according to the invention is shown in the sequence protocol under the heading SEQ ID NO. 3. The amino acid sequence of the enzyme according to the invention with a signal peptide is shown under the heading SEQ ID NO. 2. The nucleotide sequence of this enzyme with signal peptide is shown in the sequence protocol under the heading SEQ ID NO. 1. It is thus available for further developments using molecular biological methods known per se.

In another embodiment of the invention, the claimed polypeptides having perhydrolase activity have an amino acid sequence of at least 200 amino acids and, after sequence comparison by alignment with SEQ ID NO. 3 in the overlapping regions of the amino acid sequences, show at least 80% homology or at least 65% identicality to SEQ ID NO. 3, with the exception of SEQ ID NO. 3 itself. The amino acid sequence has at least 85%, more particularly at least 90%, preferably at least 95%, 96%, 96.5%, 97%, 97.5%, 98%, 98.5%, 99% and, in a most particularly preferred embodiment, 99.5% homology or at least 70%, at least 75%, at least 80%, preferably at least 85%, more particularly at least 90%, particularly preferably at least 95%, 96%, 96.5%, 97%, 97.5%, 98%, 98.5%, 99% and, in a most particularly preferred embodiment, 99.5% identicality to SEQ ID NO. 3. The polypeptides having perhydrolase activity with an amino acid sequence of at least 200 amino acids preferably have an amino acid sequence of at least 250 amino acids, more particularly at least 300 amino acids. This includes amino acid sequences which, in comparison with the described polypeptides according to SEQ ID NO. 3, have sequence regions which do not accord with one another because either they are not present in SEQ ID NO. 3, i.e. contain gaps, or have a larger number of amino acids through further insertions, but show an overlapping region with SEQ ID NO. 3. The homology of the entire amino acid sequence to be compared can thus be lower than 80% and the identicality lower than 65% by comparison with SEQ ID NO. 3, although homology should not fall below 80%, nor identicality below 65% in the overlapping region.

In the context of the present invention, an expression such as “at least X %” or “at least X %” or “X % to 99.5%” including the corner values X and 99.5 and all even-numbered and odd-numbered percentages in between . . .

Many proteins are formed as so-called pre-proteins or pre-/pro-proteins, i.e. together with a signal peptide and/or propeptide. A signal peptide is understood to be the N-terminal part of the protein of which the function is remove the protein formed from the producing cell in the periplasm or the surrounding medium. The signal peptide is then split off from the rest of the pro-protein under natural conditions by a signal peptidase. The pro-peptide ensures the correct folding of the protein and is split off from the mature, catalytically active protein. For technical applications, the mature peptides, i.e. the enzymes processed after their production, are preferred to the pre-/pro-proteins by virtue of their enzymatic activity. Pro-proteins are inactive progenitors of proteins. Their precursors with signal sequence are referred to as pre-/pro-proteins. SEQ ID NO. 3 corresponds to the sequence of the mature peptide and, with a signal peptide consisting of 16 amino acids, is described in this application as SEQ ID NO. 2. In the prior art, it is known as a polypeptide with lipase/acyl transferase activity from EP 1 275 711.

In another embodiment of the present invention, the polypeptides with perhydrolase activity have an amino acid sequence which has at least 80% homology to the amino acid sequence shown in SEQ ID NO. 3 in the positions equivalent to positions 170 to 380.

The present invention also encompasses polypeptides with perhydrolase activity and an amino acid sequence which has at least 80% homology to the amino acid sequence shown in SEQ ID NO. 3 in the positions equivalent to positions 1 to 200 or 330 to 450. In this case, too, homology is preferably at least 83%, at least 85%, more preferably at least 90%, most preferably at least 95% and, in one most particularly preferred embodiment, 99.5%, but always with the exception of SEQ ID NO. 3. In another particularly preferred embodiment, at least one amino acid is homologous in the position equivalent to position 180, 332 and/or 365 of SEQ ID NO. 3. In another particularly preferred embodiment, at least two amino acids are homologous in the positions equivalent to positions 180, 332 and/or 365 of SEQ ID NO. 3. In another particularly preferred embodiment, three amino acids are homologous in the positions equivalent to positions 180, 332 and/or 365 of SEQ ID NO. 3.

In another embodiment of the present invention, the polypeptides with perhydrolase activity have an amino acid sequence which has at least 65% identicality to the amino acid sequence shown in SEQ ID NO. 3 in the positions equivalent to positions 170 to 380. A 96% homology or a 96% identicality is particularly preferred for the part-regions in the positions equivalent to 170-190, 200 to 270 and 310 to 380.

The present invention also encompasses polypeptides with perhydrolase activity and an amino acid sequence which has at least 65% identicality to the amino acid sequence shown in SEQ ID NO. 3 in the positions equivalent to positions 1 to 200 or 330 to 450. In this case, too, identicality is preferably at least 70%, at least 75%, at least 80%, preferably at least 85%, more preferably at least 90%, most preferably at least 95% and, in one most particularly preferred embodiment, 99.5%, but always with the exception of SEQ ID NO. 3.

In another particularly preferred embodiment, at least one amino acid is identical in the position equivalent to position 180, 332 and/or 365 of SEQ ID NO. 3. In another particularly preferred embodiment, at least two amino acids are identical in the positions equivalent to positions 180, 332 and/or 365 of SEQ ID NO. 3. In another particularly preferred embodiment, three amino acids are identical in the positions equivalent to positions 180, 332 and/or 365 of SEQ ID NO. 3.

In one particular embodiment of the invention, the polypeptides are characterized by at least one modification equivalent to a position in the amino acid sequence SEQ ID NO. 3 which is selected from the group consisting of: Gly26, Ser27, Ile28, Leu29, Lys30, Thr31, Arg32, Val34, Asn36, Pro37, Leu38, Thr39, Asn40, Val41, Phe42, Thr43, Pro44, Lys46, Val47, Gln48, Asn49, Ala50, Trp51, Gln52, Leu53, Leu54, Val55, Arg56, Ser57, Asp59, Thr60, Lys79, Leu82, Val83, Ser84, Tyr85, Gln86, Thr87, Phe88, Glu89, Asp90, Ser91, Cys96, Pro98, Ser99, Tyr100, Tyr119, Thr132, Asp134, Tyr135, Pro138, Gln147, Ala151, Asn164, Leu176, Trp177, Gly178, Tyr179, Gly181, Gly182, Ser183, Leu184, Ala185, Ser186, Gly187, Trp188, Ala189, Glu199, Gly206, Ala207, Ala208, Leu209, Gly210, Gly211, Phe212, Val213, Thr214, Asn215, Ile216, Thr217, Glu221, Ala222, Asp224, Ser233, Asp245, Leu257, Leu258, Ser259, Ile260, Thr261, Tyr262, Arg263, Leu264, Gly265, Thr267, His268, Cys269, Leu270, Leu271, Asp272, Gly273, Phe278, Phe283, Ser284, Arg285, Ile286, Ile287, Arg288, Tyr289, Phe290, Pro291, Asp292, Gly293, Val297, Asn298, Gln299, Glu300, Pro301, Ile302, Lys303, Thr304, Asp308, Asn309, Asp317, Leu324, Phe325, Ile326, Tyr327, His328, Gly329, Thr330, Leu331, Ala333, Ile334, Val335, Pro336, Ile337, Val338, Asn339, Ser340, Arg341, Lys342, Thr343, Gln346, Trp347, Cys348, Glu356, Tyr357, Asn358, Glu359, Asp360, Leu361, Thr362, Asn363, Gly364, Ile366, Thr367, Glu368, Ser369, Ile370, Val371, Gly372, Ala373, Pro374, Leu377, Ile380 and Ile381.

The amino acids listed here are situated at a spatial distance of less than 12 Å from the catalytic triad formed by Ser180, Asp332 and His365. These amino acids were determined on the basis of a homology modelling with the chloroperoxidase from Pseudomonas fluorescens. In the context of the present invention, modification means that at least one of these amino acids is exchanged.

In a particularly preferred embodiment of the invention, the polypeptides are characterized by at least one modification equivalent to a position in the amino acid sequence SEQ ID NO. 3 which is selected from the group consisting of: Ser27, Ile28, Leu29, Lys30, Thr31, Arg32, Pro37, Leu38, Thr39, Asn40, Phe42, Thr43, Lys46, Val47, Gln48, Asn49, Ala50, Trp51, Gln52, Leu53, Val55, Asp59, Lys79, Ser84, Tyr85, Gln86, Thr87, Phe88, Glu89, Asp90, Pro98, Ser99, Tyr100, Tyr135, Gln147, Trp177, Gly178, Tyr179, Gly181, Gly182, Ser183, Leu184, Ala185, Ser186, Gly187, Trp188, Ala207, Ala208, Leu209, Gly210, Gly211, Phe212, Val213, Thr214, Asn215, Ile216, Thr217, Leu258, Ser259, Ile260, Thr261, Tyr262, Arg263, Leu264, Thr267, His268, Cys269, Leu271, Asp272, Phe278, Phe283, Ser284, Ile286, Ile287, Arg288, Tyr289, Phe290, Pro291, Val297, Gln299, Glu300, Pro301, Ile302, Thr304, Tyr327, His328, Gly329, Thr330, Leu331, Ala333, Ile334, Val335, Pro336, Ile337, Val338, Asn339, Ser340, Arg341, Thr343, Glu356, Asn358, Glu359, Asp360, Leu361, Thr362, Asn363, Gly364, Ile366, Thr367, Glu368, Ser369, Ile370, Val371, Leu377.

The amino acids listed in this preferred group are situated at a spatial distance of less than 10 Å from the catalytic triad formed by Ser180, Asp332 and His365.

In another particularly preferred embodiment of the invention, the polypeptides are characterized by at least one modification equivalent to a position in the amino acid sequence SEQ ID NO. 3 which is selected from the group consisting of: Ile28, Leu29, Lys30, Asn49, Ala50, Trp51, Gln52, Tyr85, Gln86, Thr87, Phe88, Tyr100, Tyr135, Gly178, Tyr179, Gly181, Gly182, Ser183, Leu184, Ala185, Leu209, Gly210, Gly211, Phe212, Val213, Ile260, Thr261, Tyr262, Ile287, Arg288, Tyr289, Glu300, His328, Gly329, Thr330, Leu331, Ala333, Ile334, Val335, Pro336, Ile337, Thr362, Asn363, Gly364, Ile366, Thr367, Glu368.

The amino acids listed in this preferred group are situated at a spatial distance of less than 6 Å from the catalytic triad formed by Ser180, Asp332 and His 365.

The positions at which the polypeptide is present in glycosylated form and the degree of glycosylation are dependent on the organism producing this polypeptide. A glycosylation at a position equivalent to position Asn164 in the amino acid sequence SEQ ID NO. 3 is preferred. In the modifications mentioned, the position equivalent to position 164 in SEQ ID NO. 3 is preferably not modified.

In the context of the invention, the term “catalytic triad” is used synonymously with the term “active center” or “catalytic center” of the polypeptide and relates to that region of the polypeptide in which the bonding of the substrates and their catalytic conversion into the products take place. The catalytic center must have a high degree of substrate specificity and must be capable of forming an active transition complex with the substrate.

In a preferred embodiment of the invention, the claimed polypeptides with perhydrolase activity are structural homologs in which the active center is identical to at least one amino acid which is equivalent to positions in the amino acid sequence SEQ ID NO. 3 that are selected from the group consisting of S180, D332 and H365 of SEQ ID NO. 3.

In the context of the invention, structural homologs are understood to be the homologs in terms of the topology, i.e. the three-dimensional structure or tertiary structure, of the protein. These structural homologs can be determined by alignment of proteins using specific algorithms that are capable of predicting a similar three-dimensional structure.

The limit of a homology sequence which leads to a structural homology depends on the length of the comparing alignment. With an alignment length of 50 amino acids, i.e. where two polypeptides each with 50 amino acids are compared, a 32.3% identicality of the amino acid sequences is needed for a structural homology to be present. For a sequence comparison of 60 amino acids, an identicality of 29.1% is sufficient. The identicality limit for a sequence comparison of, or greater than, 80 amino acids, is 24.8%. These limits t(L) are taken from an analysis of thousands of alignments from the structure data bank (PDT) and can be represented by the formula: t(L)=290.15L^{-0.562}, where L was determined for the length of the amino acid sequence between 10 and 80. Where there are more than 80 amino acids, the limit is generally 25% identicality of the amino acid sequences.

Motifs:

In another embodiment of the invention, the polypeptides with perhydrolase activity contain at least one motif which has at least 50% homology to a motif selected from the group consisting of:

| | |
|---|---|
| SEQ ID NO 4: | GYSGGxxAxxWAxxxxxxYAPE, |
| SEQ ID NO 5: | GYSGGxxAxxWAxxxxxxYAPD, |
| SEQ ID NO 6: | GFSGGxxAxxWAxxxxxxYAPE, |
| SEQ ID NO 7: | GFSGGxxAxxWAxxxxxxYAPD, |
| SEQ ID NO 8: | GYSGGxxAxxWAxxxxxxYA |
| and | |
| SEQ ID NO 9: | GFSGGxxAxxWAxxxxxxYA, |

where x stands for an essential or non-essential amino acid. In the 3-letter code for example, as in the sequence protocol, this amino acid is represented as Xaa and, again, is an essential or non-essential amino acid.

These polypeptides preferably contain motifs with at least 55%, 60%, 65%, 70%, 75% or at least 80%, preferably at least 85%, more preferably at least 90%, most preferably at least 95% and, in one most particularly preferred embodiment, 99.5% homology to the motifs shown above.

In another embodiment of the invention, the polypeptides with perhydrolase activity contain at least one motif which has at least 70% identicality to a motif selected from the group consisting of:

| | |
|---|---|
| SEQ ID NO 4: | GYSGGxxAxxWAxxxxxxYAPE, |
| SEQ ID NO 5: | GYSGGxxAxxWAxxxxxxYAPD, |
| SEQ ID NO 6: | GFSGGxxAxxWAxxxxxxYAPE, |
| SEQ ID NO 7: | GFSGGxxAxxWAxxxxxxYAPD, |
| SEQ ID NO 8: | GYSGGxxAxxWAxxxxxxYA |
| and | |
| SEQ ID NO 9: | GFSGGxxAxxWAxxxxxxYA, |

where x stands for an essential or non-essential amino acid. In the 3-letter code for example, as in the sequence protocol, this amino acid is represented as Xaa and, again, is an essential or non-essential amino acid.

These polypeptides preferably contain motifs having at least 75% or at least 80%, preferably at least 85%, more preferably at least 90%, most preferably at least 95% and, in one most particularly preferred embodiment, 99.5% identicality to the motifs shown above.

Other preferred polypeptides with perhydrolase activity are those which contain motifs with the above-mentioned homologies or identicalities to the selected motifs and which are included among the polypeptides with perhydrolase activity that have an amino acid sequence with at least 80% homology to the amino acid sequence shown in SEQ ID NO. 3, with the exception of SEQ ID NO. 3, or an amino acid sequence with at least 65% identicality to the amino acid sequence shown in SEQ ID No. 3, with the exception of SEQ ID NO. 3. The preferred homologies or identicalities apply analogously to those mentioned above in regard to motifs.

Other preferred polypeptides with perhydrolase activity are those which contain motifs with the above-mentioned homologies or identicalities to the selected motifs and which are included among the polypeptides with perhydrolase activity that have an amino acid sequence of at least 200 amino acids and, after sequence comparison by alignment with SEQ ID NO. 3 in the overlapping regions of the amino acid sequences, have at least 80% homology or at least 65% identicality to SEQ ID NO. 3, with the exception of SEQ ID No. 3.

Other preferred polypeptides with perhydrolase activity are those which contain motifs with the above-mentioned homologies or identicalities to the selected motifs and which are included among the polypeptides with perhydrolase activity that have an amino acid sequence with at least 80% homology or at least 65% identicality to the amino acid sequence in SEQ ID NO. 3 in the positions equivalent to positions 170 to 380.

In one particular embodiment, the polypeptides with perhydrolase activity according to the invention are characterized in that they are polypeptides from the family of α/β-hydrolases.

In another particular embodiment, the polypeptides with perhydrolase activity according to the invention are derivatives of the above-described polypeptides obtainable by derivatization, coupling, fragmentation, deletion mutation, insertion mutation or point mutation.

Fragments are understood to be any proteins or peptides which are smaller than natural proteins, which are smaller than proteins corresponding to those of SEQ ID No. 3, but are sufficiently homologous to them in the corresponding part-sequences, or those which correspond to completely translated genes and, for example, can also be synthetically obtained. On the basis of their amino acid sequences, they can be assigned to the complete proteins in question. For example, they can assume the same structures or perform hydrolytic activities or part-activities, such as the complexing of a substrate for example. Fragments and deletion variants of starting proteins are basically the same; whereas fragments tend to represent relatively small pieces; the deletion mutants tend to lack only short regions and hence only perform part-functions.

The fragments may be, for example, individual domains or fragments which do not accord with the domains. Such fragments can be less expensive to produce, no longer have certain, possibly unfavorable characteristics of the starting molecule, such as possibly an activity-reducing regulation mechanism, or develop a more favorable activity profile. Such protein fragments can also be produced non-biosynthetically, for example chemically. Chemical synthesis can be advantageous, for example, when chemical modifications are to be undertaken after the synthesis.

By virtue of their basic similarity, polypeptides obtainable by deletion mutation may also be assigned to the fragments. Such polypeptides can largely correspond biochemically to the starting molecules or simply no longer have individual functions. This appears to be particularly appropriate, for example, in the deletion of inhibiting regions. Ultimately, the deletions can be accompanied both by specialization and by broadening of the scope of application of the protein. If perhydrolase activity in the broadest sense is thus to be maintained, modified, specified or actually achieved, the deletion variants and the fragments are proteins according to the invention; the only additional requirement in this regard is that, beyond the homologous part-sequence still present, they lie within the specified homology range to the sequence SEQ ID NO. 3.

Polypeptides obtained by insertion mutation are understood to be those which have been obtained by insertion of a nucleic acid or protein fragment into the starting sequences by methods known per se. By virtue of their basic similarity, they may be assigned to the chimary proteins. Their only difference from those proteins lies in the size ratio of the unchanged part of the protein to the size of the protein as a whole. In such insertion-mutated proteins, the percentage of foreign protein is lower than in chimary proteins.

Inversion mutagenesis, i.e. partial sequence reversal, may be regarded as a special form both of deletion and of insertion. The same applies to a re-grouping of various parts of the molecule which differs from the original amino acid sequence. It may be regarded as a deletion variant, as an insertion variant and as a shuffling variant of the original protein.

Derivatives in the context of the present invention are understood to be polypeptides of which the pure amino acid chain has been chemically modified. Such derivatizations can be carried out, for example, biologically by the host organism in conjunction with biosynthesis of the protein. Molecular biological methods can be used for this purpose. However, they may also be carried out chemically, for example by the chemical conversion of a side chain of an amino acid or by covalent bonding of another compound to the protein. Such a compound may be, for example, other proteins which are bonded to polypeptides according to the invention, for example by bifunctional chemical compounds. Derivatization is also understood to be covalent bonding to a macromolecular carrier. Modifications such as these can influence, for example, substrate specificity or the strength of bonding to the substrate or can bring about a temporary blockage of the enzymatic activity where the coupled substance is an inhibitor. This can be relevant, for example, for the duration of storage. Accordingly, another embodiment are derivatives which have been obtained by covalent bonding to a macromolecular carrier, such as for example polyethylene glycol or a polysaccharide.

In the context of the present invention, all polypeptides, enzymes, proteins, fragments and derivatives come under the generic heading of polypeptides unless they need to be explicitly addressed as such.

The enzymatic activity can be qualitatively or quantitatively modified by other regions of the polypeptide that are not involved in the actual reaction. This relates, for example, to enzyme stability, activity, the reaction conditions or substrate specificity because, on the one hand, it is not known precisely which amino acid residues of the polypeptides according to the invention actually catalyze hydrolysis, transesterification and esterification and because, on the other hand, certain individual functions cannot be definitively excluded ab initio from participation in the catalysis process. The auxiliary functions or part-activities include, for example, the bonding of a substrate, an intermediate or end product, the activation or inhibition or mediation of a regulating influence on hydrolytic activity. This may also include, for example, the formation of a structural element situated far from the active center or a signal peptide of which the function involves removal of the protein formed from the cell and/or its correct folding and without which no enzyme capable of functioning is generally formed in vivo. Overall, however, a hydrolysis, transesterification and esterification have to be catalyzed.

Proteins can also be combined into groups of immunologically related proteins through the reaction with an antiserum or a certain antibody. The members of a group are distinguished by the fact that they have the same antigenic determinant recognized by an antibody.

Another embodiment of the invention are polypeptides or derivatives which have at least four antigenic determinants in common with one of the above-mentioned polypeptides or derivatives according to the invention, i.e. which are in accordance. At least 5 to 20 accordant antigenic determinants are preferred, at least 10 to 20 accordant antigenic determinants are particularly preferred and at least 15 to 18 accordant antigenic determinants are most particularly preferred because it is not just the pure amino acid sequence of a protein, but also its secondary structure elements and its three-dimensional folding (tertiary structure) which are critical to the performance of enzymatic activities. Thus, domains clearly differing from one another in their primary structure are capable of forming spatially substantially accordant structures and thus provide for the same enzymatic behavior. Such common features in the secondary structure are normally recognized as accordant antigenic determinants by antiserums or pure or monoclonal antibodies. Proteins or derivatives structurally similar to one another can thus be detected and assigned through immunochemical cross reactions. Accordingly, the scope of protection of the present invention also includes polypeptides or derivatives which have perhydrolase activity and which can be assigned to the above-defined proteins or derivatives according to the invention possibly not through their homology values in the primary structure, but certainly through their immunochemical relationship.

In one particular embodiment, the polypeptides with perhydrolase activity according to the invention are the above-described polypeptides with a perhydrolysis to hydrolysis ratio at temperatures of 20° C. to 30° C. of greater than 0.1. A perhydrolysis to hydrolysis ratio of or greater than 0.4; 1; 1.5; 2; 2.5; 3; 3.5 or 4 is preferred. In a particularly preferred embodiment, the perhydrolysis to hydrolysis ratio is as high as possible and, more particularly, up to 9:1.

This ratio is determined, for example, in concentration series by analyzing the content of acids released by GC analysis and the content of per acids by absorption measurement after reaction with ABTS. To this end, 90 μg polypeptide are incubated with decanoic acid methyl ester at pH=6.5 with various additions of hydrogen peroxide and the concentration of peracid is determined after a reaction time of 15 minutes. In these test series, it was possible to establish that the polypeptides according to the invention with an amino acid sequence of at least 99.5% identicality to SEQ ID NO. 3 show very high stability at hydrogen peroxide concentrations of up to 11% at reaction temperatures of 30° C. or 45° C.

Particularly preferred polypeptides with perhydrolase activity are those described above which have an at least 20% higher perhydrolysis to hydrolysis ratio than the wild-type enzyme corresponding to an enzyme with an amino acid sequence having 100% identicality to SEQ ID NO. 3.

The optimum pH for the catalytic perhydrolysis reaction, as determined at 28° C., is between 3 and 10, preferably between 4 and 9, more preferably between 6 and 7.5 and most preferably 6.5. Among enzyme mutants for detergent applications, those of which the catalytic pH optimum is displaced towards higher pH values are particularly preferred. In this case, the pH optimum is between 6.5 and 11 and more particularly between 7.5 and 10.

An optimal temperature range for the perhydrolysis of the wild-type enzyme, as determined at a pH optimum of 6.5, is between 10 and 60° C. and preferably between 20 and 45° C. An optimal temperature range for catalysis of the perhydrolysis according to the invention, as determined at a pH optimum of 6.5, is between 20 and 60° C. and preferably between 30 and 45° C.

In another embodiment of the invention, polypeptides with perhydrolase activity of the type described above are characterized in that they have optimal stability and/or optimal thermal stability and/or an optimal loss of enzymatic catalysis performance and/or optimal chemical stability and/or optimal pH stability by comparison with the wild-type polypeptide with perhydrolase activity in SEQ ID NO. 2 or SEQ ID No. 3. The optimal stabilities depend upon the application system used. The self-controlling system is described in the following. The desired enzyme should not be completely stable because this would lead at high temperatures to an overly rapid and excessively high peracid concentration and hence to damage. Optimal means that sufficient peracid can be formed in the desired temperature range at enzyme concentrations of lower than 5%.

The methods for determining these properties are well-known to the expert.

Thermal stability in the present context means that the polypeptides are active over a broad range of temperatures. It is determined via half lives of the relevant activities after incubation of the enzymes. Enzymes are active over a broad pH range. Most enzymes show a certain pH value at which their activity is optimal. Many enzyme are inactive in the extremely basic or extremely acidic range. The pH stabilities are stability values which are based on a certain pH value. The pH activity optimums and pH stability optimums do not have to accord. Enzymatic and chemical stability are based on other enzymes or chemicals which could reduce the activity of the polypeptides with perhydrolase activity according to the invention. An increased stability relates to an extended or improved activity of the polypeptides according to the invention in the presence of damaging enzymes or chemicals. This includes all enzymes or chemicals which could reduce the activity of the polypeptides according to the invention.

Polypeptides according to the invention emanating from natural sources are preferred embodiments of the present invention, particularly if they originate from microorganisms, such as single-cell fungi or bacteria, because these are generally easier to handle than multicell organisms or the cell cultures derived from multicell organisms. These represent significant options for special embodiments.

Polypeptides according to the invention or derivatives from eukaryotic fungi, especially those which are able to release the secreted proteins directly into the surrounding medium, are particularly preferred.

Particularly preferred polypeptides according to the invention or derivatives are those obtainable from microorganisms belonging to species selected from the group consisting of Candida, Kluyveromyces, Geotrichum, Fusarium, Aeromonas, Debaryomyces, Arxula, Zygoascus, Emercilla, Aspergillus, Malassezia, Gibberella, Kurzmanomyces, Ustillago, Nocardia, Cordyceps, Mycobacterium, Rhodococcus, Saccharopolyspora, Burkholderia, Corynebacterium, Saccharopolyspora, Pseudomonas, Streptomyces and Pichia.

Most particularly preferred polypeptides according to the invention or derivatives are those obtainable from microorganisms selected from the group consisting of Candida parapsilosis, Candida antarctica (Trychosporon oryzae, Pseudozyma antarctica) Candida glabrata, Candida albicans, Candida maltosa, Candida tropicalis, Candida viswanathii, Issatchenkia orientalis (Candida krusei), Kluyveromyces marxianus (C. kefyr, C. pseudotropicalis), Pichia guilliermondii (Candida guilliermondii), Geotrichum candidum, Fusarium solani, Aeromonas aerophila, Debaryomyces hansenii, Arxula adeninivorans, Zygoascus hellenicus, Aspergillus fumigatus, Emercilla nidulans, Malassezia pachydermatis, Gibberella zeae, Aspergillus niger, Fusarium sporotrichioides, Kurzmanomyces sp., Ustillago maydis, Nocardia farcinica, Cordyceps bassania, Mycobacterium tuberculosis, Mycobacterium bovis, Mycobacterium avium, Rhodococcus sp., Saccharopolyspora spinosa, Burkholderia cenocepacia, Corynebacterium diphtheriae, Pseudomonas fluorescens, Saccharopolyspora pogona and Streptomyces coelicolor.

Among the polypeptides according to the invention or derivatives from Candida species, those obtainable from Candida parapsilosis or Candida albicans are preferred and those obtainable from Candida parapsilosis CBS 604 particularly preferred, because the embodiment of the enzyme according to the invention, of which the associated sequences are shown in the sequence protocol under SEQ ID NO. 2 and SEQ ID NO. 3, was originally obtained from Candida parapsilosis CBS 604.

Strains which release the polypeptide formed into the medium surrounding them are preferred for production-related reasons.

The present invention also relates to nucleic acids which code for a polypeptide with perhydrolase activity of which the nucleotide sequence has at least 80% homology to the nucleotide sequence shown in SEQ ID NO. 1, with the exception of SEQ ID NO. 1, or 65% identicality to the nucleotide sequence shown in SEQ ID NO. 1, with the exception of SEQ ID NO. 1, more particularly nucleotide sequences which have at least 80% homology or 65% identicality over the part-region which codes for the amino acids in positions equivalent to positions 170 to 380 from SEQ ID NO. 3.

The present invention also encompasses nucleic acid coding for an amino acid sequence which has at least 85%, preferably 90%, more preferably 95% and most preferably 99.5% homology to the amino acid sequence shown in SEQ ID No. 2 or SEQ ID No. 3.

The present invention also encompasses nucleic acids coding for an amino acid sequence which has at least 70%, 75% or 85%, preferably 90%, more preferably 95% and most preferably 99.5% identicality to the amino acid sequence shown in SEQ ID No. 2 or SEQ ID No. 3.

Preferred nucleic acids are those coding for an amino acid sequence which has at least 96% identicality to the amino acid sequence in SEQ ID NO. 2 equivalent to positions 190 to 390 or to the amino acid sequence in SEQ ID No. 3 equivalent to positions 170 to 380. Nucleic acids which code for one of the above-described polypeptides or derivatives are particularly preferred. The similarity range also encompasses all polypeptides of which the nucleotide sequence has at least 85%, 87.5%, 90%, 92.5%, 95%, 96%, 97%, 98%, 99% or 99.5% identicality to the nucleotide sequence shown in SEQ ID No. 1.

Nucleic acids in the context of the present invention are understood to be the molecules built up naturally from nucleotides which act as information carriers and which code for the linear amino acid sequence in proteins or enzymes. They may be present as a single strand, as a single strand complementary to that single strand or as a double strand. As the naturally more durable information carrier, the nucleic acid DNA is preferred for molecular biological work. By contrast, for carrying out the present invention in a natural environment, such as for example in an expressing cell, an RNA is formed, so that RNA molecules crucial to the invention also represent embodiments of the present invention. With DNA, the sequences of both complementary strands have to be taken into account in all three possible reading frames. Another factor to be taken into account is that various codon triplets can code for the same amino acids, so that a certain amino acid sequence of several different nucleotide sequences with possibly very little identicality can be derived (degeneracy of the genetic code). In addition, different organisms show differences in the use of this codon. For these reasons, both amino acid sequences and nucleotide sequences have to be included in the consideration of the scope of protection and stated nucleotide sequences should only be regarded as an exemplary coding for a certain amino acid sequence.

The unit of information corresponding to a protein is also referred to as a gene in the present specification. By applying now generally known methods, such as for example chemical synthesis or the polymerase chain reaction (PCR) in conjunction with molecular-biological and/or protein-chemical standard methods, the expert is now able with the aid of known DNA and/or amino acid sequences to produce the corresponding nucleic acids up to and including complete genes.

Changes to the nucleotide sequence, which can be made for example by molecular-biological methods known per se, are referred to as mutations. Depending on the type of change, there are, for example, deletion, insertion or substitution mutations or mutations where various genes or parts of genes are fused to one another (shuffling); these are gene mutations. The associated organisms are known as mutants. The proteins derived from mutated nucleic acids are known as variants. For example, deletion, insertion or substitution mutations or fusions lead to deletion-, insertion- or substitution-mutated or fusion genes and, at protein level, to corresponding deletion, insertion or substitution variants or fusion proteins.

The term “identicality” used in the present specification in relation to the amino acid sequence means an identicality to the particular amino acid sequence, so that the polypeptide with a stated identicality has the same biological activity as the first polypeptide. The identicality of the nucleotide sequence relates to a gene homologous to a first nucleotide sequence. Homologous in relation to the nucleotide sequence means that the gene can be allelic. In addition, homologous in relation to the nucleic acid sequence means that the gene can emanate from another species, although the polypeptide coded by this gene has the same biological activity as the polypeptide coded by the first nucleotide sequence.

Vectors in the context of the present invention are understood to be elements consisting of nucleic acids which contain an interesting gene as the characterizing nucleic acid region. They are capable of establishing this as a stable genetic element in a species or a cell line over several generations or cell divisions. Vectors are special plasmids, i.e. circular genetic elements, particularly when used in bacteria. In genetic engineering, a distinction is drawn on the one hand between vectors which are used for storage and hence also, as it were, for genetic work, the so-called cloning vectors, and on the other hand vectors which perform the function of producing the interesting gene in the host cell, i.e. enabling the expression of the particular protein. These vectors are known as expression vectors.

According to the present invention, the nucleic acid is suitably cloned in a vector. Accordingly, the molecular-biological dimension of the invention consists in vectors with the genes for the corresponding proteins. Such vectors can include, for example, those derived from bacterial plasmids, from viruses or from bacteriophages or predominantly synthetic vectors or plasmids with elements of diverse origin. With the other genetic elements present, vectors are able to establish themselves as stable units in the particular host cells over several generations. So far as the present invention is concerned, it is immaterial whether they establish themselves extrachromosomally as independent units or are integrated into a chromosome. Which of the many systems known from the prior art is selected will depend on the particular individual case. Determining factors can be, for example, the number of copies achievable, the selection systems available, including above all antibiotic resistances, or the cultivatability of the host cells capable of accommodating the vectors.

The vectors form suitable starting points for molecular-biological and biochemical studies of the particular gene or associated protein, for further developments of the invention and, lastly, for the amplification and production of polypeptides according to the invention. They represent embodiments of the present invention insofar as the sequences of the nucleic acid regions according to the invention which are present lie within the homology regions defined in detail in the foregoing.

Preferred embodiments of the present invention are cloning vectors. Besides for suitable for storage, biological amplification or selection of the interesting gene, cloning vectors are also suitable for characterizing the particular gene, for example through the establishment of a restriction map or sequencing. Cloning vectors are also preferred embodiments of the present invention because they represent a transportable and storable form of the claimed DNA. They are also preferred starting points for molecular-biological techniques which are not dependent on cells, such as the polymerase chain reaction for example.

Expression vectors are chemically similar to the cloning vectors, but differ in those part-sequences which enable them to replicate in the host organisms optimized for the production of proteins and to bring the gene present to expression there. Preferred embodiments of the invention are expression vectors which themselves carry the genetic elements required for expression. Expression is influenced, for example, by promoters which regulate the transcription of the gene. Thus, expression can take place through the natural promotor originally located before this gene, but also after genetic fusion both through a promotor of the host cell made available on the expression vector and through a modified or completely different promotor of another organism.

Preferred embodiments are expression vectors which can be regulated through changes to the culture conditions or addition of certain compounds, such as for example cell density or special factors. Expression vectors enable the associated protein to be produced heterologously, i.e. in an organism different from that from which it can naturally be obtained. The scope of protection of the present invention also encompasses the homologous production of proteins from a host organism expressing the gene via a suitable vector. This can have the advantage that natural modification reactions associated with the translation can be carried out on the protein formed in exactly the same way as they would take place naturally.

Other embodiments of the present invention can also be cell-free expression systems where the protein biosynthesis is completed in vitro. Such expression systems are also established in the prior art. The in vivo synthesis of an enzyme according to the invention, i.e. through living cells, requires the transfer of the associated gene to a host cell—its so-called transformation. In principle, suitable host cells are any organisms, i.e. prokaryotes, eukaryotes or archaea.

The present invention also relates to a vector which contains a nucleic acid region according to the invention and which can be, for example, a cloning vector or an expression vector and to a host cell which expresses, or can be stimulated to express, one of the polypeptides according to the invention or derivatives, preferably using an expression vector according to the invention. In principle, suitable host cells are any organisms, i.e. prokaryotes, eukaryotes or cyanophyta. Preferred host cells are those which are genetically easy to handle, for example with regard to transformation with the expression vector and its stable establishment, for example single-cell fungi or bacteria. In addition, preferred host cells are distinguished by favorable microbiological and biotechnological handling behavior. This includes, for example, easy cultivation, high growth rates, minimal fermentation media requirements and good production and secretion rates for foreign proteins. The optimal expression systems often have to be experimentally determined for each individual case from the large number of various known systems available. Each protein according to the invention can thus be obtained from a plurality of host organisms.

Preferred embodiment are host cells which can be regulated in their activity through genetic regulation elements which are available, for example, on the expression vector or which may even be present in these cells from the outset. For example, these host cells can be stimulated to express by controlled addition of chemical compounds, such as methanol for example, which act as activators, by changing the cultivation conditions or on reaching a certain cell density. This provides for highly economic production of the interesting proteins.

A variant of this experimental principle are expression systems where additional genes, for example those made available on other vectors, influence the production of proteins according to the invention. Such genes can be modifying gene products or those which are to be purified together with the protein according to the invention, for example in order to influence its function. Such products can include, for example, other proteins or enzymes, inhibitors or elements which influence the interaction with various substrates.

Preferred host cells are prokaryotic or bacterial cells. Bacteria are generally distinguished from eukaryotes by shorter generation times and less stringent requirements with regard to the cultivation conditions. Inexpensive processes for producing proteins according to the invention can be established in this way. Particularly preferred host cells are host cells, more particularly bacteria, which secrete the protein or derivative formed into the surrounding medium, so that the expressed proteins according to the invention can be directly purified. Heterologous expression is preferred. Gram-positive bacteria, such as actinomycetes or bacilli for example, have no outer membrane, so that they release the secreted proteins directly into the medium surrounding them. Accordingly, preferred bacteria for heterologous expression are those of the genus Bacillus, more particularly those of the species listed below. Gram-negative bacteria may also be used for heterologous expression. In their case, a large number of proteins are secreted into the periplasmatic space, i.e. into the compartment between the two membranes enclosing the cells. This can be advantageous for special applications. Such bacteria include, for example, those of the genus Klebsiella or Escherichia, preferably of the species listed below. Eukaryotic cells can also be suitable for the production of polypeptides according to the invention. Examples of such cells are yeasts, such as Saccharomyces or Kluyveromyces. This can be particularly advantageous, for example, when the proteins—in connection with their synthesis—are to undergo specific modifications which such systems allow. These include, for example, the binding of low molecular weight compounds, such as membrane anchors or oligosaccharides.

Microorganisms particularly preferred for the production of polypeptides according to the invention from transformed host cells are microorganisms selected from the group consisting of Candida parapsilosis, Saccharomyces cerevisiae, Schizosaccharomyces pombe, Pichia pastoris, Pichia boidinii, Pichia stipitis, Hansenula polymorpha, Kluyveromyces lactis, Schwanniomyces castellii, Yarrowia lipolytica, Escherichia coli, Bacillus subtilis, Bacillus amyloliquifaciens, Bacillus stearothermophilus, Bacillus licheniformis, Lactococcus lactis, Streptococcus lactis, Lactobacillus bulgaricus, Aspergillus orizae, Aspergillus niger, Trichoderma reesei, Mucor sp and Rhizopus sp.

The transformed host cells, also known as transformants, are then cultivated in known manner and the polypeptides according to the invention are isolated. The host cells of the process according to the invention are cultivated and fermented in known manner, for example in batch or continuous systems. In the first case, a suitable nutrient medium is inoculated with the organisms and the product is harvested from the medium after a period of time to be experimentally determined. Continuous fermentations are distinguished by the reaching of a flow equilibrium in which cells partly die off, but also regrow, over a comparatively long period of time and, at the same time, product can be removed from the medium. Fermentation processes are well known from the prior art and represent the actual industrial-scale production step, followed by a suitable purification method. Any fermentation processes based on one of the above-mentioned processes for producing the recombinant proteins represent correspondingly preferred embodiments of this aspect of the present invention. The particular optimal conditions for the production processes used, for the host cells and/or the proteins to be produced have to be experimentally determined using the knowledge of the expert, for example in regard to fermentation volume, media composition, oxygen supply or stirring speed, in conjunction with the previously optimized cultivation conditions for the particular strains.

The perhydrolases according to the invention can be made available in the quantity required for industrial use by cloning and expression.

All the elements discussed in the foregoing can be combined into processes for producing polypeptides according to the invention. Accordingly, the present invention also relates to such processes. A broad range of combinations of process steps is possible for each protein according to the invention. They all put into effect the idea on which the present invention is based, namely quantitatively producing a protein type defined via the perhydrolase activity and, at the same time, the high homology to the sequences shown in the sequence protocols with the aid of the associated genetic information. The optimal process has to be experimentally determined for each actual individual case. More particularly, the present invention relates to a process for the production of a polypeptide or derivative as described above using a nucleic acid which codes for an amino acid sequence that has at least 80% homology to the amino acid sequence in SEQ ID NO. 3 and/or using a vector and/or using a transformed host cell as described above or using a cell which this naturally forms.

The present invention also relates to uses of the polypeptides according to the invention with perhydrolase activity together with hydrogen peroxide which, in the particular use, can also be produced in situ by corresponding agents.

A critical factor is the in situ formation of the peracid by the enzymatic reaction. It is very important that a self-controlling system is involved. The higher the temperature and the higher the concentration of per acid, the more aggressive the effect of the peracid. Accordingly, the system should release less peracid at relatively high temperatures (50-60° C.). This is achieved by the enzyme showing optimal stability or instability. Under the laws of physics, with increasing temperature, there is an increase in the reaction rate and hence in the concentration of the peracid released. With increasing concentration or activity of the perhydrolase, there is also an increase in the quantity of peracid formed per unit of time. At relatively high temperatures, however, the enzyme is partly inactivated. Inactivation proceeds more quickly, the higher the temperature. Now, in this perhydrolysis system, use is made of the opposing effects of peracid formation at increasing temperature. By thermally inactivating the enzymes through the increase in temperature, the formation of peracid is limited, so that excessively high, damaging peracid concentrations can be avoided.

A local overconcentration of peracid on the article to be treated is also avoided by the fact that the peracid reaction can only take place when both the perhydrolase is thoroughly dispersed or dissolved and the other components, such as ester and hydrogen peroxide, are available at the same time. This is the case when all components are finely dispersed or dissolved.

Overall, the system leads to a limited concentration of peracids. Damage by bleaching reaction on the fabric is thus prevented. The enzyme has to be sufficiently stable to all other components in the detergent and also shows sufficient stability to hydrogen peroxide.

In the present specification, the term “peracid” is used synonymously with “peroxo acid”.

The enzyme produces peracids, for example in situ, from fatty acid esters under the catalytic effect of the reaction of the fatty acid ester with hydrogen peroxide. The hydrogen peroxide required is formed from hydrogen peroxide sources or by oxidases present. The fatty acid esters from fatty acids with a chain length of C7 to C12 are particularly suitable because they have a particularly high available oxidation potential and can be obtained from renewable raw materials.

The peracids formed in situ can be used in another reaction with olefins for the production of epoxides.

A preferred embodiment of the use according to the invention is the use in cosmetic or pharmaceutical preparations, the use in laundry detergents or cleaning products and/or the use for bleaching, for dye transfer inhibition and for disinfection.

The polypeptides with perhydrolase activity according to the invention may advantageously be introduced into body care preparations, hair shampoos, hair care preparations, oral hygiene, dental care or denture care preparations, cosmetics, laundry detergents, cleaning preparations, after-rinses, hand washing preparations, manual dishwashing detergents, automatic dishwasher detergents, disinfectants and preparations for the bleaching or disinfecting treatment of filter media, textiles, pelts, paper, skins or leather, for the disinfecting or bleaching or oxidizing treatment of surfaces and solutions. Accordingly, the present invention also relates to the preparations mentioned containing a polypeptide with perhydrolase activity as described above.

A preferred embodiment of the invention is a laundry detergent or bleaching preparation containing a bleaching system, which is capable of producing highly active available oxygen with the aid of hydrogen peroxide under in-use conditions, and optionally synthetic surfactant, organic and/or inorganic builder and other typical constituents of bleaching preparations or laundry detergents, characterized in that the bleaching system consists of a polypeptide with perhydrolase activity according to the invention, a substrate for the perhydrolase (fat precursor), a hydrogen peroxide source or a hydrogen-peroxide-producing system, such as oxidase.

Available oxygen (A.O) in the context of the invention is understood to be the oxidatively active available oxygen which is present in the oxidizing agent (for example H₂O₂). The oxidizing agent H₂O₂ contains, for example, one mol available oxygen per mol H₂O₂. Highly active available oxygen in the context of the invention is understood to be available oxygen with a higher oxidation potential than the available oxygen of the starting compound. For example, hydrogen peroxide is reacted with available oxygen to form a peracid containing available oxygen. However, the peracid has a higher oxidation potential than hydrogen peroxide, so that it contains “highly active available oxygen”.

Other preferred embodiments of the invention are the above-mentioned preparations which additionally contain other additives. These other additives are preferably selected from the group consisting of surfactants, builder material, builders, complexing agents, softeners, dispersants, bleach activators, solid inorganic and/or organic acids or acidic salts, dyes, perfumes, pigments, redeposition inhibitors, dye transfer inhibitors and foam inhibitors or mixtures thereof.

Other preferred embodiments of the invention are the above-mentioned preparations which, in addition to the bleaching system, contain
5% by weight to 70% by weight and, more particularly, 10% by weight to 50% by weight surfactant,
10% by weight to 65% by weight and, more particularly, 12% by weight to 60% by weight water-soluble, water-dispersible inorganic builder material
1% by weight to 10% by weight and, more particularly, 2% by weight to 8% by weight water-soluble inorganic builders,
not more than 15% by weight solid inorganic and/or organic acids or acidic salts,
not more than 5% by weight heavy metal complexing agent,
not more than 5% by weight redeposition inhibitor,
not more than 5% by weight dye transfer inhibitor and
not more than 5% by weight foam inhibitor.

The bleaching system is characterized in that it is based on an available oxygen carrier. For example, sodium perborate contains ca. 10% available oxygen (A.O); pure hydrogen peroxide contains 47% available oxygen. The bleaching system may contain 5 to 95% by weight of an available oxygen carrier, preferably 10 to 60% by weight. This corresponds to a preferred available oxygen content of 1 to 6% A.O in the system. The bleaching system additionally contains a substrate which is described in the following.

According to the invention, the preparations mentioned contain 0.00001 to 3% by weight of the polypeptides according to the invention as described and defined above, expressed as active substance and based on 100% pure active catalyst, based on the total quantity of preparation, including water and optionally the additives mentioned added together to 100%.

The perhydrolase is normally used as a formulated enzyme. The active substance typically amounts to between 0.05 and 10.0% of the formulated end product although this is not intended to limit the invention in any way. The active substance can also be directly used for the purposes of the invention.

A quantity of 0.00001 to 2% by weight active substance is preferred, a quantity of 0.00005 to 0.3% by weight active substance being particularly preferred. The formulated perhydrolase is preferably used in quantities of 0.01 to 50% by weight and more preferably in quantities of 0.1 to 10% by weight.

The substrate for the perhydrolase (fat precursor) is preferably formed by a compound corresponding to general formula (I):
R¹Oₓ[(R²)ₘ(R³)ₙ]ₚ (I)
where
(i) R¹ is selected from the group consisting of H, substituted or unsubstituted alkyl, heteroalkyl, alkenyl, alkynyl, aryl, alkylaryl, alkyl heteroaryl or heteroaryl groups, primary, secondary, tertiary or quaternary amine radicals,
(ii) R² is an alkoxyl group,
(iii) R³ is an ester-forming group with the formula (II) R⁴CO—, where R⁴ represents H, alkyl, alkenyl, alkynyl, aryl, alkylaryl, alkyl heteroaryl or heteroaryl groups,
(iv) x=1 where R¹ is H and, where R¹ is not H, x has a value smaller than or equal to the number of carbon atoms in R¹,
(v) p has a value smaller than or equal to x,
(vi) m is an integer of 0 to 50 and
(vii) n is an integer with a value of at least 1.

The substrate is preferably selected from fatty acid esters with a fatty acid residue of 6 to 14 C atoms or from fatty acid esters of polycarboxylic acids, more particularly C12 dicarboxylic acid, or especially selected from methyl, ethyl or glycerol esters of butyric, caproic, octanoic or decanoic acid, preferably caproic acid methyl ester, octanoic acid methyl ester, trioctanoin and, in a particularly preferred embodiment, readily biodegradable esters: triglycerides, partial glycerides, methyl and ethyl esters with chain lengths of C7 to C12. The quantity of substrate for the perhydrolase present in the detergent according to the invention is dependent on the quantity of percarboxylic acid required to obtain the desired bleaching result and can be adjusted as required by the expert.

The oxidases are preferably selected from the group consisting of aldose oxidase, galactose oxidase, cellobiose oxidase, pyranose oxidase, verbose oxidase, hexose oxidase, glucose oxidase and mixtures thereof.

The preparations preferably contain a hydrogen peroxide source selected from the group consisting of alkali metal percarbonates, perphosphates, persulfates, organic peracids and urea/hydrogen peroxide or mixtures thereof and especially selected from sodium percarbonate, sodium perborate or potassium monopersulfate triple salt, salt mixtures of percarbonate/potassium persulfate in a ratio of 1:3 (pH 5.5); percarbonate/potassium persulfate in a ratio of 1:1 (pH 7.5); percarbonate/potassium persulfate in a ratio of 3:1 (>pH 9) and, in a particularly preferred embodiment, H₂O₂.

A polypeptide with perhydrolase activity according to the invention is preferably used in such quantities that the preparation as a whole has a perhydrolase activity of 0.05 to 5 ppm AO/μg and preferably 0.2 to 1.2 ppm AO/μg. Preparations with perhydrolase activities in the ranges mentioned have a sufficiently rapid percarboxylic acid release for standard European automatic washing processes whereas the increase in the quantity of perhydrolase present to higher activities does not generally produce a commensurately high increase in bleaching performance.

A polypeptide with perhydrolysis activity according to the invention is preferably used in such quantities that percarboxylic acid is released sufficiently quickly under in-use conditions. In addition, a continuous supply of the percarboxylic acid over the period of use is also desirable to maintain sufficiently high active concentrations of the percarboxylic acid formed. In this connection, the formation of 0.1 to at most 100 ppm available oxygen of the percarboxylic acid of a dilute solution over the period of use is desirable. The increase in the amount of perhydrolase present to higher activities does not generally result in higher bleaching performance levels. The perhydrolase is preferably used in such quantities that the ratio by weight of enzyme (expressed as active substance) to theoretically possible available oxygen formation of the percarboxylic acid is in the range from 100:1 to 1:500 and preferably in the range from 10:1 to 1:100.

Laundry detergents and cleaning preparations include any form of detergents for fabrics, for example in powder form, in gel form or in the form of a liquid detergent. They also include all possible cleaning preparations which may be used for the bleaching or disinfection of fabrics, such as clothing, bed linen, curtains and furnishing materials and for the disinfection of surfaces.

The cosmetic and/or pharmaceutical preparations may be present, for example, in the form of foam baths, shower baths, creams, gels, lotions, alcoholic and aqueous/alcoholic solutions, emulsions, wax/fat compounds, stick preparations, powders or ointments and may contain the polypeptides with perhydrolase activity according to the invention. These cosmetic and/or pharmaceutical preparations may also contain mild surfactants, oil components, emulsifiers, superfatting agents, pearlizing waxes, consistency factors, thickeners, polymers, silicone compounds, fats, waxes, stabilizers, biogenic agents, deodorants, antiperspirants, antidandruff agents, film formers, swelling agents, UV protection factors, antioxidants, hydrotropes, preservatives, insect repellents, self-tanning agents, solubilizers, perfume oils, dyes and the like as further auxiliaries and additives.

According to the invention, the preparations mentioned are preferably characterized in that they are present as tablets, granules or free-flowing powders with a bulk density of 300 g/l to 1200 g/l and, more particularly, 500 g/l to 900 g/l. In addition, the preparations may advantageously be present in the form of a paste-like or liquid detergent.

In addition, besides the additives mentioned either individually or collectively, the preparations according to the invention may also contain enzyme stabilizers and/or other enzymes, more particularly proteases, amylases, cellulases, hemicellulases, cutinases, pectinases, mannanases, endoglycosidases, lysozymes, cell-wall-degrading enzymes, other oxidoreductases and/or lipases.

The present invention also relates to the use of the preparations mentioned with individual, several or all of the other embodiments mentioned in cosmetic or pharmaceutical preparations, in laundry detergents or cleaning compositions and/or for bleaching, dye transfer inhibition and for disinfection.

### EXAMPLES

### Example 1

### Determination of Enzyme Activity

Hydrolytic activity (standard units) was tested by mixing 1 ml of an enzyme extract in 50 mM phosphate buffer pH 6.5. with 10 μmol decanoic acid methyl ester (in 50 μl acetone). The reaction was carried out with stirring for 15 mins. at 30° C. in closed Wheaton vessels and was stopped by addition of 0.95 ml ethanol/H₂SO₄ (100/0.8). 50 μl of an internal standard consisting of 1.5 μmol lauric acid methyl ester (C12 methyl ester) and 1.5 μmol lauric acid were added to the solution.

A comparison was prepared by mixing ethanol/H₂SO₄ to the solution without enzyme.

The fats were extracted with 1 ml hexane and analyzed by GC after silylation. To this end, 25 μl MSTFA ((N-methyl-N-trimethylsilyl)-trifluoroacetamide) and 25 μl pyridine were added to 200 μl hexane extract. The mixture was incubated for 20 mins. at 50° C. before being injected into the GC. The GC (HP 5890 II with Autosampler) is equipped with a J&W DB1 column (30 m, 0.32 mm diameter, 0.1 column μm film thickness), a split injector and an FID and Waters Millennium 32 acquisition and integration software. The vector gas was helium (28 ml/min.). The decanoic acid methyl ester and the decanoic acid were analyzed with lauric acid methyl ester and lauric acid as internal standard.

Results: the perhydrolase used showed an activity of 9.7 μmol released decanoic acid per minute per mg perhydrolase at 30° C.

### Example 2

### Peracid Determination

The peracid content was determined using ABTS (2,2′-azino-bis-(ethylbenzothioazoline-6-sulfonic acid) as indicator. The reaction medium consisted of 100 μl solution to be analyzed, 200 μl acetic acid1 M, 50 μl Kl 100 mg/l, 550 μl water and 100 μl ABTS 1 g/l. The reaction was started by adding ABTS to the solution to be analyzed. The peracid to be determined oxidizes ABTS to the radical which then appears as blue in color. Absorption was determined after incubation for 10 mins. at room temperature at a wavelength of 405 nm. The standard curve was obtained through different concentrations of solutions to be analyzed.

Calibration was carried out by photometric absorption measurement of the ABTS reaction product using defined concentrations of peracetic acid.

Under these test conditions, 1 μmol/ml peracetic acid corresponds to an extinction of 0.75.

### Example 3

### Perhydrolysis Activity and Selectivity of the Enzyme

The enzyme reaction medium contained (in a volume of 1 ml) 10 μmol decanoic acid methyl ester, 250, 500 and 1000 ppm H₂O, 2 μg enzyme (perhydrolase with an amino acid sequence at least 99.5% identical to SEQ ID NO. 3) and phosphate buffer pH 6.5, 50 mM. The reaction was carried out at 30° C. and 45° C. and was started by adding the enzyme (perhydrolase with an amino acid sequence at least 99.5% identical to SEQ ID NO. 3). The reaction was stopped after 15 mins. by addition of 1 ml ethanol.

A comparison was prepared by replacing the enzyme with water. The same conditions were applied. Ethanol was also added to the comparison in order to be able to determine a zero point.

Since the perhydrolase forms both acid and peracid, a total acid content and a peracid content were determined. The selectivity of the reaction can be determined from the two measurements.

TABLE 1
Perhydrolase Activity

| | Enzyme | | Acid + | | | Total | Fatty acid | |
|---|---|---|---|---|---|---|---|---|
| | solution | Sub- | Peracid | Peracid | | hydrolysis | hydrolysis | Perhydro- |
| Tem- | μg | strate | after 15 | after 15 | | activity | activity | lysis activity |
| per- | Protein/ | C10Me | mins. | mins. | H₂O₂ | μmol/min/ | μmol/min/ | μmol/min/ |
| ature | ml | μmol | μmol/ml | μmol/ml | (ppm) | mg | mg | mg |
| 30° C. | 2 | 10 | 0.29 | 0.04 | 250 | 9.7 | 8.3 | 1.3 |
| | | | 0.29 | 0.213 | 500 | 9.7 | 2.6 | 7.1 |
| | | | 0.29 | 0.2 | 1000 | 9.7 | 3.0 | 6.7 |
| 45° C. | 2 | 10 | 0.58 | 0.02 | 250 | 19.3 | 18.7 | 0.7 |
| | | | 0.68 | 0.22 | 500 | 22.7 | 15.3 | 7.3 |
| | | | 0.57 | 0.18 | 1000 | 19.0 | 13.0 | 6.0 |

TABLE 2
Selectivity of the enzyme at 30° C. and 45° C.

| | | Fatty acid | | |
|---|---|---|---|---|
| | Hydrolysis | hydrolysis | Perhydrolysis | |
| H₂O₂ | activity | activity | activity | Peracid/acid |
| (ppm) | μmol/min/mg | μmol/min/mg | μmol/min/mg | selectivity |
| 30° C. | | | | |
| 250 | 9.67 | 8.33 | 1.33 | 0.16 |
| 500 | 9.67 | 2.57 | 7.10 | 2.77 |
| 1000 | 9.67 | 3.00 | 6.67 | 2.22 |
| 45° C. | | | | |
| 250 | 19.33 | 18.67 | 0.67 | 0.04 |
| 500 | 22.67 | 15.33 | 7.33 | 0.48 |
| 1000 | 19.00 | 13.00 | 6.00 | 0.46 |

The Example shows that selectivity reaches >2.5 at 30° C./500 ppm H₂O₂. At higher temperatures where excessive concentrations of peracid can be damaging to the material to be treated, selectivity advantageously falls below 0.5, even at relatively high H₂O₂ concentrations. The goal of a self-controlling system is thus achieved.

### Example 4

### Enzyme Stability and Activity Test in the Presence of Hydrogen Peroxide

For the stability test, 90 μg enzyme (perhydrolase with an amino acid sequence at least 99.5% identical to SEQ ID NO. 3) were incubated with stirring in closed vessels for 0, 15, 30 and 60 minutes at 30° C. or 45° C. in the presence of various concentrations of H₂O₂. An enzyme activity test was then carried out with these enzyme solutions in the same way as in Example 1.

TABLE 3
Result of activity determination (μmol/min/mg) at 30° C.
after pre-incubation without H₂O₂ and with various H₂O₂
concentrations Evaluation is carried out by putting the
enzyme activity at 0 mins. incubation at 100% and evaluating
stability from the residual activities.

| H₂O₂ in % | 0 min. | 15 mins. | 30 mins. | 60 mins. |
|---|---|---|---|---|
| 0 | 4.7 | 4.5 | 4.1 | 4.1 |
| 2.25 | 5.2 | 3.7 | 4.1 | 4.6 |
| 11.25 | 7.0 | 3.8 | 4.4 | 5.7 |
| 16.9 | 11.8 | 3.4 | 3.9 | 5.0 |
| 22.5 | 8.2 | 3.4 | 2.9 | 2.1 |

TABLE 4
Residual activity at 30° C., pre-incubation with hydrogen peroxide

Residual activity after pre-incubation
at 30° C. in minutes

| H₂O₂ in % | 0 | 15 | 30 | 60 |
|---|---|---|---|---|
| 0.00 | 100% | 96% | 87% | 87% |
| 2.25 | 100% | 71% | 79% | 88% |
| 11.25 | 100% | 54% | 63% | 81% |
| 16.90 | 100% | 29% | 33% | 42% |
| 22.50 | 100% | 41% | 35% | 26% |

TABLE 5
Result of activity determination (μmol/min/mg) at 45° C.

| H₂O₂ in % | 0 min. | 15 mins. | 30 mins. | 60 mins. |
|---|---|---|---|---|
| 0 | 8.1 | 6.9 | 6.2 | 6.1 |
| 2.25 | 7.2 | 7.1 | 6.0 | 5.2 |
| 11.25 | 6.8 | 7.0 | 5.3 | 4.1 |
| 16.9 | 6.8 | 2.2 | 1.7 | 0.3 |
| 22.5 | 2.3 | 0.1 | 0 | 0 |

TABLE 6
Residual activity at 45° C., pre-incubation with hydrogen peroxide

Residual activity after pre-incubation
at 45° C. in minutes

| H₂O₂ in % | 0 | 15 | 30 | 60 |
|---|---|---|---|---|
| 0.00 | 100% | 85% | 77% | 75% |
| 2.25 | 100% | 99% | 83% | 72% |
| 11.25 | 100% | 103% | 78% | 60% |
| 16.90 | 100% | 32% | 25% | 4% |
| 22.50 | 100% | 4% | 0% | 0% |

The enzyme according to the invention shows high stability at H₂O₂ concentrations of up to 11%, more particularly at 30° C. At higher reaction temperatures, stability deteriorates relatively quickly at concentrations above 11%.

### Example 5

### Bleaching Tests

1.9 mg decanoic acid methyl ester in the form of a PIT emulsion (PIT=phase inversion temperature) were reacted with 550 ppm hydrogen peroxide and 9 μg perhydrolase/ml at pH 6.5/45° C.

The same solution without the perhydrolase was used for comparison.

These mixtures were applied for 2, 15 and 60 minutes to fabric samples stained with red wine or yellow marker pen. After only 15 minutes, no difference could be seen in the bleaching effect against red wine and yellow marker pen. After 60 mins., bleaching was far more effective with than without enzyme. The stains were almost completely removed with enzyme.

1. Polypeptides having perhydrolase activity with an amino acid sequence which has at least 80% homology to the amino acid sequence shown in SEQ ID NO. 3 with the exception of SEQ ID NO. 3.

2. Polypeptides having perhydrolase activity of claim 1 having an amino acid sequence which has at least 65% identicality to the amino acid sequence shown in SEQ ID NO. 3 with the exception of SEQ ID NO. 3.

3. Polypeptides having perhydrolase activity with an amino acid sequence of at least 200 amino acids which, after sequence comparison by alignment with SEQ ID NO. 3 in the overlapping regions of the amino acid sequences, have at least 80% homology to SEQ ID NO. 3 with the exception of SEQ ID NO. 3.

4. Polypeptides having perhydrolase activity of claim 3 having an amino acid sequence of at least 200 amino acids which, after sequence comparison by alignment with SEQ ID NO. 3 in the overlapping regions of the amino acid sequences, have at least 65% identicality to SEQ ID NO. 3 with the exception of SEQ ID NO. 3.

5. Polypeptides having perhydrolase activity with an amino acid sequence which is at least 80% homologous to the amino acid sequence shown in SEQ ID NO. 3 in the positions equivalent to positions 170 to 380 with the exception of SEQ ID NO. 3.

6. Polypeptides having perhydrolase activity of claim 5 having an amino acid sequence which is at least 65% identical to the amino acid sequence shown in SEQ ID NO. 3 in the positions equivalent to positions 170 to 380 with the exception of SEQ ID NO. 3.

7. Polypeptides having perhydrolase activity as claimed in claim 1, characterized in that they are structural homologs where the active center is identical to at least one amino acid which is equivalent to positions in the amino acid sequence of SEQ ID NO. 3 that are selected from the group consisting of S180, D332 and H365 of SEQ ID No. 3.

8. Polypeptides with perhydrolase activity containing at least one motif which is at least 70% homologous to a motif selected from the group consisting of

| | |
|---|---|
| SEQ ID NO 4: | GYSGGxxAxxWAxxxxxxYAPE, |
| SEQ ID NO 5: | GYSGGxxAxxWAxxxxxxYAPD, |
| SEQ ID NO 6: | GFSGGxxAxxWAxxxxxxYAPE, |
| SEQ ID NO 7: | GFSGGxxAxxWAxxxxxxYAPD, |
| SEQ ID NO 8: | GYSGGxxAxxWAxxxxxxYA |
| and | |
| SEQ ID NO 9: | GFSGGxxAxxWAxxxxxxYA. |

9. Polypeptides with perhydrolase activity of claim 8 containing at least one motif which is at least 50% identical to a motif selected from the group consisting of

| | |
|---|---|
| SEQ ID NO 4: | GYSGGxxAxxWAxxxxxxYAPE, |
| SEQ ID NO 5: | GYSGGxxAxxWAxxxxxxYAPD, |
| SEQ ID NO 6: | GFSGGxxAxxWAxxxxxxYAPE, |
| SEQ ID NO 7: | GFSGGxxAxxWAxxxxxxYAPD, |
| SEQ ID NO 8: | GYSGGxxAxxWAxxxxxxYA |
| and | |
| SEQ ID NO 9: | GFSGGxxAxxWAxxxxxxYA. |

10. Nucleic acids coding for a polypeptide with perhydrolase activity, of which the nucleotide sequences are at least 80% homologous to the nucleotide sequence shown in SEQ ID NO. 1, with the exception of SEQ ID NO. 1.

11. Nucleic acids coding for a polypeptide with perhydrolase activity of claim 10, of which the nucleotide sequences are at least 65% identical to the nucleotide sequence shown in SEQ ID NO. 1, with the exception of SEQ ID NO. 1.

12. A vector, more particularly a cloning vector or expression vector, which contains a nucleic acid according to claim 10.

13. A transformed host cell, more particularly host cells from microorganisms, which expresses or can be stimulated to express one of the polypeptides or derivatives according to claim 1.

14. Body care preparations, hair shampoos, hair care preparations, oral hygiene, dental care or denture care preparations, cosmetics, laundry detergents, cleaning preparations, after-rinses, hand washing preparations, manual dishwashing detergents, automatic dishwasher detergents, disinfectants and preparations for the bleaching or disinfecting treatment of filter media, textiles, pelts, paper, skins or leather, for the disinfecting or bleaching or oxidizing treatment of surfaces and solutions, containing the polypeptide with perhydrolase activity of claim 1.

15. A laundry detergent or bleaching preparation containing a bleaching system, which is capable of producing percarboxylic acid under in-use conditions, and optionally synthetic surfactant, organic and/or inorganic builder and other typical constituents of bleaching preparations or laundry detergents, characterized in that the bleaching system contains the polypeptide with perhydrolase activity of claim 1, a hydrogen peroxide source or a hydrogen-peroxide-generating system, such as oxidase, and a substrate for the perhydrolase (fat precursor).

16. A transformed host cell, more particularly host cells from microorganisms which expresses or can be stimulated to express one of the polypeptides or derivatives according to claim 8.

17. A transformed host cell, more particularly host cells from microorganisms which expresses or can be stimulated to express one of the polypeptides or derivatives according to claim 10.
